(19) United States
(12) Patent Application Publication
(10) Pub. No.: US 2009/0062731 A1
(43) Pub. Date: Mar. 5, 2009

(54) Breast Pump

(75) Inventors: Cheng Keyong, Wenzhou City (CN); Richard S. Weston, Carlsbad, CA (US)

Correspondence Address: PROCOPIO, CORY, HARGREAVES & SAVITCH LLP, 530 B STREET, SUITE 2100, SAN DIEGO, CA, 92101, US

(73) Assignee: HYGEIA II MEDICAL GROUP, INC., Carlsbad, CA (US)

(21) Appl. No.: 12/168,056

(22) Filed: Jul. 3, 2008

(30) Foreign Application Priority Data
Jul. 4, 2007 (CN) 200720169558.8

(51) Int. Cl. A61M 1/06

(52) U.S. Cl. 604/74

(57) ABSTRACT

The breast pump shield assembly includes a breast shield having a parabolic shape with upper and lower apertures. A conduit in fluid communication with the lower aperture receives the expressed milk and directs it to a container. A vacuum introduces through the upper aperture creates a suction within the breast shield. A center insert fits closely within the lower aperture and has a flared end with an annular surface for contacting and applying an outward force against a circular area around the nipple. A stepped-down distal section of the center insert has an outlet end with a flap valve to prevent suction from being drawn into the breast milk container while permitting expressed milk to flow into the container.

### RELATED APPLICATIONS

This application claims the priority of Chinese Utility Model Application No. 200720169558.8, filed Jul. 4, 2007 in the State Intellectual Property Office of the People's Republic of China, the disclosure of which is incorporated herein by reference.

### TECHNICAL FIELD OF THE INVENTION

The present application relates to a breast pump for use by lactating women and more particularly to a breast pump shield assembly for use with a vacuum source.

### BACKGROUND OF THE INVENTION

Breastfeeding is advocated worldwide because the nutrients in mother's milk are rich, well-balanced and easy to digest and absorb for infants, all qualities that cannot be fully replicated by processed formulas. In situations where breastfeeding is not possible, for example, where the mother has returned to a regular work routine or is otherwise temporarily separated from the infant, it is necessary to express milk from the breasts for storage and use during the period of separation. Also, if the infant is unable to effectively draw out the milk, for example, due to premature birth, illness or underdeveloped nursing reflexes, it may necessary to express the milk from the mother's breast using a breast pump. The most commonly available manual breast pumps on the market generally include a sucking bottle and a suction source. A funnel-shaped breast cover is positioned over the opening of the sucking bottle. The suction source is used to generate negative pressure within the bottle to draw out the milk. The inadequacies of many existing breast pumps include that: 1) they rely solely on a sucking function, which can cause pain or discomfort to the breasts; 2) the mother's milk is expressed only under the effect of negative pressure inside the funnel-shaped breast cover without compression of the nipples, so that the nipples and areolas are stretched and deformed.

Some prior art systems have attempted to simulate the compressive force that is generated by an infant's mouth (tongue, lips and palate) by applying an intermittent compression around the nipple. One such example is described in U.S. Pat. No. 7,166,087 of Silver et al. In this system, bladder inserts within the breast shield are intermittently inflated and deflated to produce a massaging effect. However, the basic principle is the same as other prior art systems—the nipple is drawn by suction toward the apex of the funnel-shaped breast shield, stretching and deforming the nipple.

### SUMMARY OF THE INVENTION

An object of the present invention is to provide a breast pump that utilizes a combination of sucking and compressing functions to virtually eliminate the pain that can occur during suction alone, and minimize deformation of the nipples. Furthermore, since the inventive breast pump shield assembly can press outward against the areola encircling the nipple while it draws out the mother's milk, simulating some of the motions used for manual expression, the volume of milk produced is increased.

In an exemplary embodiment, the breast pump includes a bottle or other container and a breast pump shield assembly in fluid communication with the bottle. In order to perform simultaneous sucking and extruding functions, a center insert and vacuum-generating means, e.g., a pump diaphragm, a pull rod and a handle for manual pumping, are provided. Other mechanisms may be used to generate the suction, including other types of manual pumps and electric pumps as are known in the art.

In one embodiment, the breast pump shield assembly includes a breast shield having a parabolic shape with upper and lower apertures. A conduit in fluid communication with the lower aperture receives the expressed milk and directs it to a container. A vacuum introduces through the upper aperture creates a suction within the breast shield. A center insert fits closely within the lower aperture and has a flared end with an annular surface for contacting and applying an outward force against a circular area surrounding the nipple and areola. A stepped-down distal section of the center insert has an outlet end with a flap valve to prevent suction from being drawn on the breast milk collection container while permitting expressed milk to flow into the container.

In one aspect of the invention, the breast pump shield assembly includes a breast shield with an open proximal end for receiving a breast to form a substantially air-tight seal around the breast and a distal end for communicating expressed milk to a breast milk collection container, the distal end having an extraction aperture and a suction aperture disposed above the extraction aperture with a partition disposed between the two apertures. A conduit extends from the distal end of the breast shield in fluid communication with the extraction aperture for receiving the expressed milk and directing it to the container. The conduit includes a flap valve to prevent suction from being drawn on the container while permitting expressed milk to flow into the container. A vacuum source in communication with the suction aperture creates suction within the breast shield. In a preferred embodiment, the breast shield is generally parabolic in shape to allow expansion of the breast when negative pressure is applied to the breast. A center insert disposed within the extraction aperture has a cylindrical center section with an outer diameter sized to closely fit within the central aperture, a flared proximal end with an annular surface for contacting and applying an outward force against a circular area around a nipple and areola area of the breast, and a stepped-down distal section with an outlet end extending into the conduit. The stepped-down distal section has an interior volume much smaller than an interior volume of the cylindrical center section to assist in the flow of breast milk to the collection container. The center section may include an opening formed through an upper sidewall thereof so that the interior volume of the cylindrical center section is in fluid contact with the vacuum source. Alternatively, a separate vacuum source may be connected to the interior volume of the center insert to apply suction directly to the nipple area. In the preferred embodiment, the valve is formed integrally in the center insert near its distal end.

In another aspect of the invention, the breast pump shield assembly includes a breast shield having an open proximal end for receiving a breast to form a substantially air-tight seal around the breast and a distal end with an aperture for communicating suction to an interior of the breast shield. A conduit extends from the distal end of the breast shield in fluid communication with the aperture for receiving the expressed milk and directing it to a container. At least one vacuum source in communication with the aperture creates suction within the breast shield. A center insert extends outward from the aperture toward the proximal end of the breast shield. The center insert has a cylindrical center section with an outer diameter sized to closely fit within the aperture, a flared proximal end with an annular surface for contacting and applying a counterforce against a circular area around a nipple and areola of the breast, and a stepped-down distal section with an outlet end extending into the conduit. The stepped-down distal section has an interior volume much smaller than an interior volume of the cylindrical center portion and a valve formed near the outlet end to prevent air from being drawn from the container while permitting expressed milk to flow into the container. The center section has an opening formed through an upper sidewall thereof for introducing suction into the interior volume of the center section. When a vacuum is applied to the breast shield and the center insert, the breast and nipple are drawn in toward the distal end of the breast shield while the proximal end of the center insert opposes the inward force to press outward against the breast in the circular area around the nipple so that milk is expressed into and through the center insert and out into the container.

In still another aspect of the invention, the breast pump shield assembly includes a breast shield having a parabolic shape generally conforming to a shape of a breast with an open proximal end for receiving and forming a substantially air-tight seal around the breast and a distal end for communicating expressed milk to a container. The distal end of the breast shield includes at least one aperture. At least one vacuum source in communication with the at least one aperture creates a suction within the breast shield. A center insert extends outward from the at least one aperture toward the proximal end of the breast shield, the center insert having a cylindrical center section with an outer diameter sized to closely fit within an interior of the aperture, a flared proximal end with an annular surface for contacting and applying a counterforce against a circular area around a nipple of the breast, and a stepped-down distal section with an outlet end extending into the conduit. The stepped-down distal section has an interior volume much smaller than an interior volume of the cylindrical center portion and a valve formed near the outlet end to prevent air from being drawn from the container while permitting expressed milk to flow into the container. The center section has an opening formed through an upper sidewall thereof so that the interior volume is in fluid contact with the at least one vacuum source. In a preferred embodiment, the at least one aperture comprises two apertures separated by a partition, with a suction aperture disposed above an expression aperture.

In a manual breast pump embodiment, the breast shield includes an upper shell, which defines a vacuum chamber, a lower shell separated from the upper shell, which acts as a conduit, and a side shell connected via apertures with each of the upper and lower shells. The side shell is formed as a parabolic bowl-shape that generally conforms to the shape of a breast. Different sizes of the parabolic side shell, both depth and diameter, may be selected to provide a better fit to an individual's breast size. Inside the side shell are formed an upper, suction aperture communicating through a peripheral wall of the upper shell and a lower, expression aperture communicating through a peripheral wall of the lower shell. In the exemplary embodiment, the upper aperture has an arcuate shape, partially surrounding the lower aperture. The breast shield may be formed from of a polycarbonate, polypropylene or other rigid plastic material.

A handle support shaft extends upward from the back wall of the upper shell so that it extends to a height above the upper edge of the upper shell. A handle pivot is formed near the upper extent of the support shaft for pivotable attachment of the handle. The lower shell has an internal thread at its lower opening for mating with the external threads of a bottle or other container.

The center insert is a hollow stepped cylinder with three sections: a flared (trumpet-shaped) proximal end that is configured to contact and comfortably apply an outward force (opposing the suction that draws the breast into the breast shield) across a circular area around the nipple, a center section which has an outer diameter that closely fits within the lower aperture and a distal stepped-down section. The stepped-down section is formed with an open channel to define a continuous channel from the proximal end to the distal end terminating at an openable cover that serves as a one-way valve to prevent air from being drawn from the container while permitting milk to flow into the container. An opening is formed through the upper wall of the center section near its proximal end to provide communication between the suction source and the interior of the center insert so that a vacuum is created within the interior of the center insert.

For manual pumping, the flexible diaphragm is a cup-like shell with a central opening and an upper lip which forms an annular channel that fits closely over the upper edge of the upper shell to form a substantially air-tight seal between the diaphragm and the upper shell. The profile of the diaphragm generally matches the profile of the upper shell when the diaphragm is in a relaxed position. The center insert and the flexible diaphragm are preferably made of silicone gel.

The pull rod includes, from bottom to top, a bottom plate, a lower flange, a cylindrical portion, an upper flange for defining the range of movement of the handle relative to the pull rod, and a knob at the top of the pull rod. The upper surface of the bottom plate abuts the lower surface of the flexible diaphragm with the cylindrical rod passing through the central opening of the diaphragm to provide a substantially air-tight seal, so that lifting and lowering of the bottom plate lifts and lowers the center of the diaphragm, respectively. The pull rod has a variable length to adjust the range of motion of the diaphragm, thus increasing or decreasing the amount of suction created by the pumping action.

The inverted hook-shaped handle is pivotably mounted on the handle pivot of the handle support shaft. A fork extends from a front portion of the handle to bracket the pull rod near the upper flange to transfer upward and downward motion to the pull rod, thus raising and lowering the center of the flexible diaphragm.

In one embodiment, the at least one vacuum source has separate connections to each of the breast shield and the collection container. Alternatively, separate pumps may be used. By producing a separate suction within the collection bottle, splashback is eliminated by positively drawing the expressed milk from the center insert into the bottle to minimize the risk of contamination. In still another embodiment, separate connections or separate pumps may be provided for the breast shield and the center insert.

To enable operation using a technique known as “draw and hold” and to facilitate storage of the breast pump for travel, the handle can be locked with the handle in a depressed position, i.e., with the pull rod and diaphragm center lifted to their highest travel. A window is formed on the handle near the handle pivot through which extends a rectangular tab for controlling a pivoting latch fastener. With the handle in the depressed position, pressing down on the tab pivots the latch fastener around an axle to swing a pair of hooks upward to capture a pair of pegs extending laterally from the handle support shaft, below the handle pivot. Lifting the tab upwards releases the latch fastener.

Using prior art systems, a mother would have to have to hold the suction in place to allow suction to be distributed across the breast and nipple area. As the mother begins pumping, rapid strokes are needed to stimulate the breast to begin the letdown process. After the flow of milk is established, a slower pump stroke can be used or alternatively the lock mechanism can be activated to “hold” the pump mechanism in the active suction position. This is a unique aspect of the present invention which reduces the amount of energy exerted by the mother and will likely make this technique more popular.

A bottle adapter may be provided between the bottle and the breast shield so that the breast shield assembly can be used with bottles of different sizes. The bottle adapter is machined with both internal and external threads so as to engage with an external thread provided at an opening of the bottle and with an internal thread of a lower shell of the breast shield, respectively.

A removable dust cover may be provided to fit over the front of the side shell of the breast shield to prevent contaminants from entering the breast shield when it is not in use.

In comparison with the prior art, the present invention provides the following advantageous effects: (1) combined sucking function and expressing functions better simulate the sucking rhythm of infants to eliminate the pain generated during milk suction and effectively avoid crushing the nipples; (2) since the breast pump simultaneously uses suction and gentle compression to draw out the mother's milk so that, the volume of the expressed milk is increased, and the milk is cleaner; (3) the center insert uniquely provides a counteracting force on the nipple and areola to more effectively remove milk; (4) the pump locking tab enables the pump to be locked in an active position to enable the pump to continue to provide suction without the mother needing to use physical force and energy to remove milk from the breast (5) the center insert has a valve integrally formed therein, thus reducing the number of pieces in the pump, making the pump simpler and easier to clean; (6) the pump uses a unique variable piston rod to change the amount of pressure that the mother can apply with one stroke of the suction source; (7) the pump uses an off center suction aperture to enable suction to be applied to be breast tissue versus directly on the nipple and areola.

### BRIEF DESCRIPTION OF THE DRAWINGS

FIG. 1 is a schematic perspective view of the present invention;

FIG. 2 is a schematic cross sectional view of the embodiment in FIG. 1;

FIG. 3 is a schematic perspective view of a breast cover according to the present invention;

FIG. 4 is a schematic perspective view of a center insert according to the present invention;

FIG. 5 is a schematic perspective view of a flexible diaphragm according to the present invention;

FIG. 6 is a schematic perspective view of a pull rod according to the present invention; and

FIG. 7 is an enlarged schematic perspective view of a latch fastener according to the present invention.

FIG. 8 is a cross-sectional view of the breast shield assembly positioned over a breast for expression of milk.

FIG. 9 is a cross-sectional view of an alternate embodiment of the invention for use with an electric pump.

FIG. 10 is a cross-sectional view of an alternate embodiment of the invention for use with an electric pump.

### DETAILED DESCRIPTION OF THE INVENTION

The present invention will be described in further detail with reference to an exemplary embodiment in conjunction with the drawings. However, the scope of the present invention is not intended to be limited to the exemplary embodiment.

The reference signs of the parts used in the drawings are as follows:

1: base, 10: bottle adapter, 20: 29: aperture partition, bottle, 30: breast shield, 31: upper shell (vacuum chamber), 32: lower shell (conduit), 33 side shell (breast cover), 34: partition, 35: breast cover, 36: upper (vacuum) aperture, 37: lower (liquid) aperture, 38: handle support shaft, 39: handle pivot, 40: center insert, 41: proximal end (nipple cover), 42: stepped-down section, 43: through channel, 44: flap valve, 45: vacuum opening, 46: center section, 50: dust cover, 60: flexible diaphragm, 61: center hole, 62: upper lip, 70: pull rod, 71: bottom plate, 72: lower flange, 73: center cylindrical portion, 74: upper flange, 75 knob, 76: lower cylindrical portion, 77: upper cylindrical portion, 80: latch fastener, 81 rectangular tab, 82: latch pivot, 83: hook, 90: handle, 91: fork, 92: pivot bracket, 93: window, 94: latch pivot loop.

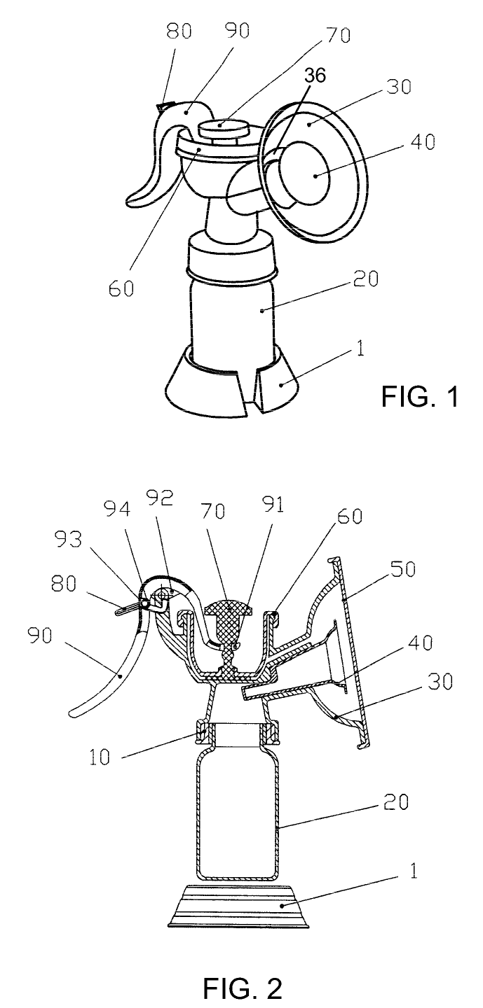

As shown in FIGS. 1 and 2, a manual breast pump includes a bottle 20, a base 1 removably disposed below the bottle 20 and a breast shield 30 disposed above the bottle 20. A center insert 40 is removably disposed within an aperture 37 within the breast shield 30. For manual production of a vacuum, a pull rod 70 is connected to a flexible diaphragm 60 at one end and a handle 90 at the other end to produce a vacuum within an upper portion of the breast shield. A bottle adapter 10 may be provided between the bottle 20 and the breast shield 30 so that the breast shield 30 can be adapted for use with bottles 20 of different sizes. The bottle adapter 10 is machined with internal and external threads so as to engage with an external thread provided at an opening of the bottle 20 and with an internal thread of a lower shell 32 of the breast shield 30, respectively.

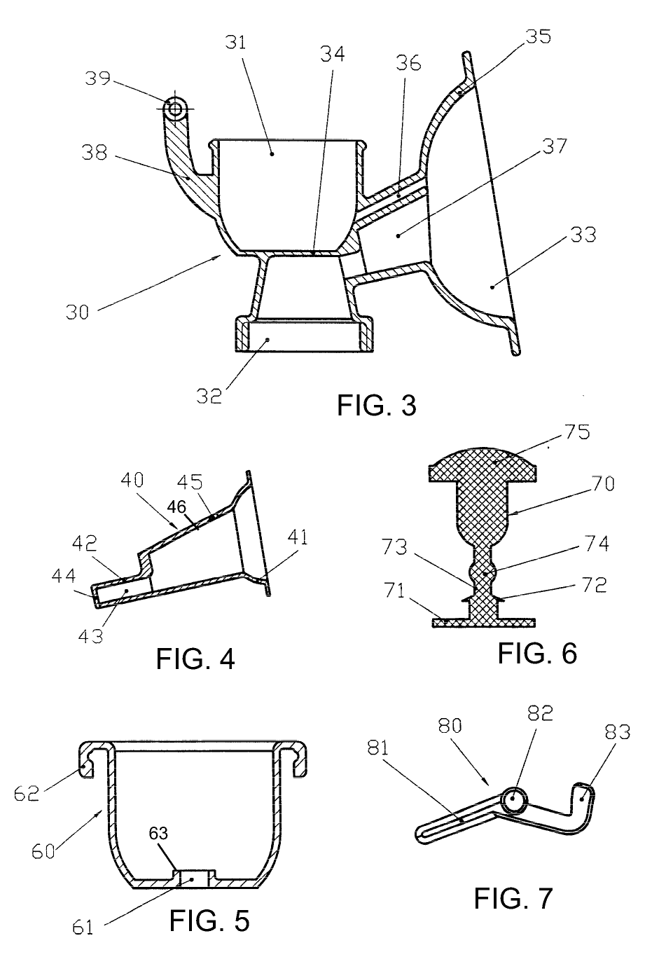

As shown in FIG. 3, the breast shield 30 for the exemplary manual embodiment includes an upper shell 31, a lower shell 32, and a side shell 33 connected to the upper and lower shells via apertures. A partition 34 is formed between the upper shell 31 and the lower shell 32, thus isolating the upper shell 31, which defines the chamber in which suction is generated, from the lower shell 32, which serves as the conduit through which expressed milk is directed into the bottle 20. The side shell 33, i.e., breast cover, is formed as an open bowl-shaped parabolic trumpet 35 that allows the breast to be drawn into the interior volume when suction is applied. The open, proximal edge of the side shell 33 is formed with a generally smooth, rounded surface to enhance comfort as the edge is pressed against the breast to produce a substantially air-tight seal within the interior of the shell. Different sizes of parabolic side shell, both in terms of depth and diameter, may be selected to provide a better fit to the mother's breast size. Inside the side shell 33 at its distal side are formed an upper, suction aperture 36 which provides a vacuum connection communicating through a peripheral wall of the upper shell 31 and a lower, liquid aperture 37 through a peripheral wall of the lower shell or conduit 32, separated by a partition 29. In the exemplary embodiment, the partition 29 is formed in a downward arc so that the suction aperture 36 has an arcuate shape that partially encircles liquid aperture 37, as illustrated in FIG. 1. Upper aperture 36 should have a large enough cross-sectional area to introduce sufficient vacuum into the chamber to produce an appropriate level of suction. Lower aperture 37 is generally off-center, lower than the actual central focus of the parabolic shell. In an alternate embodiment, a single aperture may be used for both suction and liquid if the center insert described below is used. In general, the apertures should be large enough to facilitate cleaning, preferably without requiring special tools.

A handle support shaft 38 extends upward at an angle from the back peripheral wall of the upper shell 31, terminating at a height above the upper edge of the upper shell 31. A handle pivot 39 is formed near the upper extent of the support shaft 38 for pivotable attachment of the handle 90. The lower shell 32 is formed with an internal thread at its lower opening for mating with the external threads of a bottle 20 or other container. The breast shield 30 is preferably formed from a molded polycarbonate material. To prevent the breast shield 30 from becoming contaminated, a removable dust cover 50 may be fitted over the proximal opening of the side shell 33 of the breast shield 30.

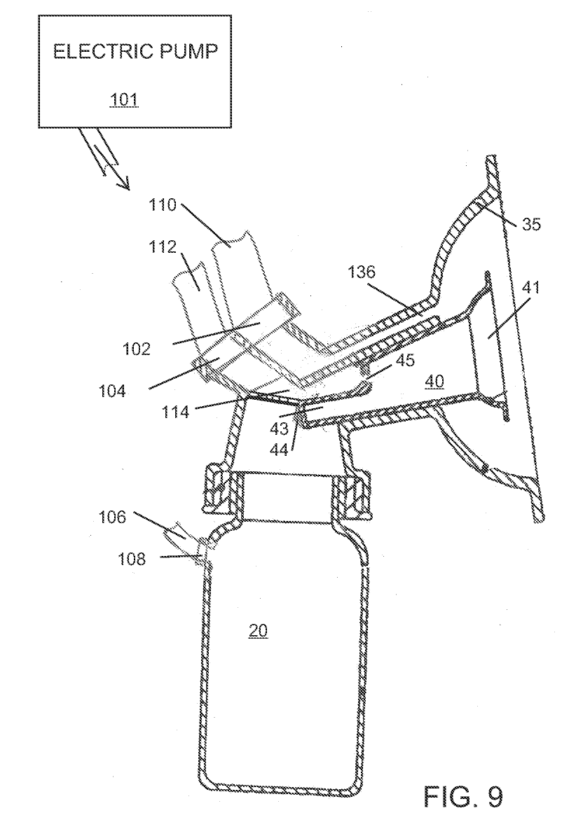

As shown in FIG. 4, the center insert 40 is a hollow stepped cylinder with three sections: a flared (trumpet-shaped) proximal end 41 which contacts a circular area around the nipple. The curvature and finish of the proximal end 41 is selected for comfortably contact and apply an outward force against the breast surrounding the nipple and at least a portion of the areola. The profile of the center insert 40 generally matches that of the lower aperture 37 of the breast shield 30. A center section 46 of center insert 40 has an outer diameter that closely fits within the lower aperture 37, as shown in FIG. 2. An opening 45 is formed through the upper portion of center section 46 to provide a fluid communication to the vacuum source for introducing suction into the interior volume of center insert 40. As illustrated, opening 45 is near the proximal end 41 to enable access to the upper aperture 36. Note that if a separate vacuum source is used for producing suction within the center insert 40, the opening will not be positioned to communicate with the upper aperture 36 and instead will be located more toward the distal end of the center section to provide access to a separate vacuum source, such as a vacuum port. (See, e.g., FIG. 9.) A rear stepped-down section 42 is formed with a open through-channel 43 to define a continuous channel from the proximal end 41 to an outlet at the distal end of stepped-down section 42. An openable flap valve 44 is located at the outlet end of the stepped portion 42. The flap valve 44 is pulled closed when suction is applied to the breast shield 30, but permits milk to flow into the conduit (lower shell) 32 and into the container 20. The center insert 40 is preferably formed from silicone gel.

As shown in FIG. 5, the flexible diaphragm 60 is a cup-like shell with a center opening 61 through its lower surface, surrounded by bottom flange 63, and an upper lip 62 which forms an annular channel 62 that closely fits over the upper edge of upper shell 31 to form a substantially airtight seal between the lower surface of the diaphragm to and the upper surface of upper shell 31. The flexible diaphragm 60 is made of silicone gel.

As shown in FIG. 6, the pull rod 70 includes, from bottom to top, a bottom plate 71, a lower cylindrical portion 76 with an outer diameter to pass through and closely fit the inner diameter of center opening 61 of diaphragm 60, a lower flange 72 which retains bottom flange 63 against the upper surface of the bottom plate 71, a center cylindrical portion 73, an upper flange 74, an upper cylindrical portion 77 and a knob 75 at the top of the pull rod 70. The combination of the upper flange 74, the upper cylindrical portion 77 and the lower portion of knob 75 provide define the area of the pull rod 70 that cooperates with the handle 90 to raise and lower the diaphragm. In a preferred embodiment, the center cylindrical portion 73 has a variable length to allow the throw of the pumping action to be adjusted. For example, the center cylindrical portion 73 can be threaded to allow the upper flange 74, upper cylindrical portion 77 and knob 75 to be screwed up or down to extend the effective length of center cylindrical portion 73.

As shown in FIG. 2, the inverted hook-shaped handle 90 is pivotably retained on the handle pivot 39 on the handle support shaft 38. A fork 91 extends from a front portion of the handle 90 to bracket the upper cylindrical portion 77 of the pull rod 70 between the lower portion of knob 74 and the upper flange 74. A pivot bracket 92 extends from the inner surface of the handle 90 to capture and pivot around handle pivot 39. When the lower end of handle 90 is pressed inward, the handle pivots around handle pivot 39 to raise the fork 91, applying an upward force against the lower portion of knob 74 to lift the pull rod 70 and the flexible diaphragm 60, thus generating suction within upper shell 31. When the lower end of handle 90 is pulled outward, the fork 91 is pivoted downward, pressing against upper flange 74 to push the pull rod 70 and the flexible diaphragm downward, forcing air out of the upper shell 31.

To facilitate operation of the manual pump using the “draw and hold” technique, and to enable compact storage of the breast pump, the handle 90 can be locked in a depressed position, with the pull rod and diaphragm raised to their highest travel. A window 93 is formed in the handle 90 near the handle pivot 39. As shown in FIG. 7, a latch fastener 80 has a latch pivot 82 which is supported within a latch pivot loop 94 that spans the window 93 between its upper and lower edges. One end of the latch fastener has two arc-shaped hooks arranged symmetrically along the centerline of the latch fastener 80 for capturing corresponding pegs extending laterally from the handle support shaft 38. A rectangular tab 81 extends through the window 93 for pivoting the latch pivot 82. With the handle 90 in the depressed position, the rectangular tab 81 is pressed downward with a thumb or finger to swing the latch fastener 80 around latch pivot 82, causing the hook(s) 83 to lift upward to capture the peg(s) on the support shaft 38, locking the handle in position. By lifting tab 81 upward, the hooks 83 release the pegs and allow the handle 90 to be lifted and depressed freely.

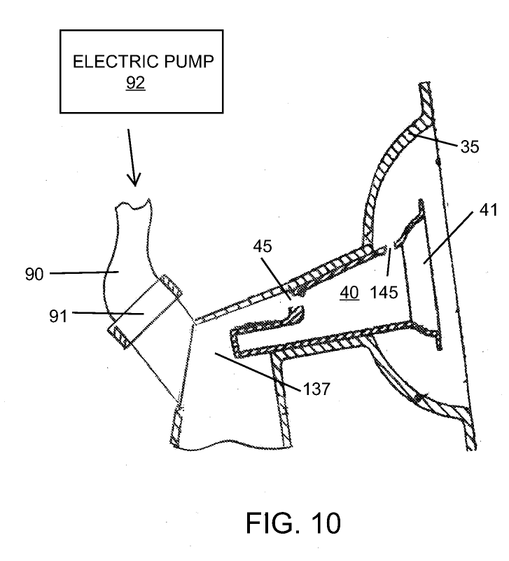

In an alternative embodiment, the components of the manual pump, i.e., the upper shell 31, the flexible diaphragm 60, pull rod 70, latch fastener 80 and handle 90, can be replaced with an electric pump to produce the appropriate suction to be introduced through the suction aperture 36. Such pumps are generally well known in the art. FIG. 10 illustrates an embodiment of the inventive breast pump assembly for use with an electric pump 92 as the vacuum source. In this embodiment, a single aperture 137 takes the place of the separate upper and lower apertures described above. The center insert 40 fits closely within the inner diameter of aperture 137 and suction is drawn on the interior volume of the center insert 40 through opening near the distal end of the center section of center insert 40. A second opening 145 formed through the upper sidewall of center insert 40 near proximal end 41 communicates the suction to the interior volume of breast shield 35. Vacuum port 91 is connected near the distal end of aperture 137 and is connected via vacuum tubing 90 to electric pump 92. As in the previously described embodiments, aperture 137 serves as a conduit for directing expressed milk into a container below (not shown).

FIG. 9 illustrates an alternative embodiment of the invention with a number of additional features that may be incorporated separately or in various combinations into the inventive breast pump assembly described above. The individual features illustrated include the use of an electric pump 101 instead of the manual pump. While the electric pump 101 may be connected to aperture 136 to function in a similar manner to the manual pump where all suction is generated through aperture 36, as illustrated, separate pump connection ports 102 and 104 are provided for independently creating suction through aperture 136 to breast cover 35, to draw the breast into the parabolic shell, and within center insert 40, to draw the nipple and a portion of the areola into center insert 40. In this arrangement, the opening 45 for communicating vacuum to the interior of the center insert 40 is located near the distal end of the center section adjacent to separate vacuum aperture 114, thus isolating the two separate suction volumes defined by the aperture 136 with breast cover 35 and the aperture 114 with center insert 40. A single vacuum line from pump 101 can be connected to both connection ports 102 and 104, or separate lines 110 and 112, as shown, from either the same pump or from two separate pumps can be used. A third vacuum port 108 may be provided near the top of the collection container 20 to produce a suction within the container to actively draw the liquid through the outlet end 43 of center insert 40 instead of passively relying on gravity alone. Vacuum port 108 can be connected by vacuum line 106 to the same pump 101 or to a separate pump. By actively drawing the liquid from the center insert 40, the risk of splashback is virtually eliminated, thus reducing the risk of contamination of the milk.

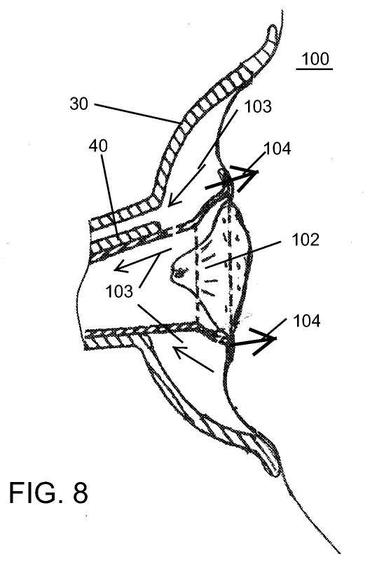

The operation principle of the present invention is described with reference to FIGS. 2 and 8. To express the mother's milk, the parabolic side shell 35 is aligned with the breast 100 and the center insert 40 aligned with the nipple 102 at its center, then pressed against the breast 100 to form a substantially air-tight seal within both the breast cover 30 and the center insert 40. The suction source is activated to draw the breast 100 and nipple (with at least a portion of the areola) 102 inward, as indicated by arrows 103 in FIG. 8, which causes the breast tissue and the nipple/areola to expand to fill the parabolic shell. Using the manual pump embodiment, the suction is generated by pressing the handle 90 to move the pull rod 70 upward. The flexible diaphragm 60 it pulled upward under the effect of the bottom plate 71 of the pull rod 70. The suction draws air within the breast cover 30 and center insert 40 (via vacuum opening 45) through upper aperture 36 into the space formed by the flexible diaphragm 60 and the upper shell 31. As the breast 100 and nipple 102 are drawn by the suction into the breast cover 30 and center insert 40, respectively, the proximal end (nipple cup) 41 of center insert 40 applies an opposing force (arrows 104) in a circular area around the nipple 102 to generate a compressive force against the expanded breast and nipple/areola to simulate the combined suction and compression produced by an infant's sucking motion, expressing the milk from the breast. When the handle 90 is released, the suction is discontinued, allowing the expressed milk to flow through channel 43 of the center insert 40, passing by the flap valve 44 to be collected in bottle 20. Thus, an operation cycle is completed. As previously described, it may be necessary to perform a series of rapid pumping cycles to encourage the letdown reflex before the milk will begin to flow. Thereafter, the pumping rate may be reduced. Where an electric pump is being used, the cycle is initiated by pressing a switch or turning a knob to activate the vacuum source.

The breast pump according to the present invention provides the advantage of combining sucking functions with compressing functions. Since the breast cover and center insert can compress the breast and nipple area while it draws out the mother's milk by simulating manual expression, the expressed milk is cleaner and the volume is increased.

While the present invention has been described herein with reference to the preferred embodiments thereof, it will be apparent to those skilled in the art that general modifications and substitutions can be made without departing from the scope of the invention.

1. A breast pump assembly comprising:
a breast shield having an open proximal end for receiving a breast to form a substantially air-tight seal around the breast and a distal end for communicating expressed milk to a container, the distal end having two apertures formed therein with a partition therebetween, the two apertures comprising a lower aperture and an upper aperture;
a conduit extending from the distal end of the breast shield in fluid communication with the lower aperture for receiving the expressed milk and directing it to the container, the conduit comprising a valve to prevent suction from being drawn on the container while permitting expressed milk to flow into the container; and
at least one vacuum source in communication with the upper aperture for creating a suction within the breast shield.

2. The breast pump assembly as in claim 1, wherein the partition between the lower and upper apertures is arcuate so that the upper aperture partially encircles the central aperture.

3. The breast pump assembly as in claim 1, further comprising a center insert disposed within the lower aperture, the center insert comprising a cylindrical center section with an outer diameter sized to closely fit within the lower aperture, a flared proximal end having an annular surface for contacting and applying an outward force against a circular area around a nipple of the breast, and a stepped-down distal section having an outlet end extending into the conduit, the stepped-down distal section having an interior volume much smaller than an interior volume of the cylindrical center section, the center section having an opening formed through an upper portion thereof so that the interior volume of the cylindrical center section is in fluid contact with the at least one vacuum source.

4. The breast pump assembly as in claim 3, wherein the valve is integrally formed in the center insert at an end of the interior volume at the stepped-down distal section.

5. The breast pump assembly as in claim 3, wherein the center insert comprises a silicone gel.

6. The breast pump assembly as in claim 1, wherein the vacuum source is a manual pump comprising a vacuum chamber in fluid communication with the upper aperture, a flexible diaphragm fitted within and sealing the vacuum chamber, and a handle for moving a center portion of the flexible diaphragm into and out of the vacuum chamber to generate a vacuum within the vacuum chamber.

7. The breast pump assembly as in claim 6, wherein the handle further comprises a locking mechanism for locking the flexible diaphragm in a raised position.

8. The breast pump assembly as in claim 6, further comprising a pull rod having a lower end attached to the center portion of the flexible diaphragm and an upper end attached to a knob, wherein the handle cooperates with the knob to raise and lower the pull rod.

9. The breast pump shield assembly as in claim 8, wherein the pull rod has a variable length for adjusting a range of travel of the lower end of the pull rod.

10. The breast pump assembly as in claim 1, wherein the breast shield has a parabolic shape.

11. The breast pump assembly as in claim 1, wherein the at least one vacuum source is an electric pump.

12. The breast pump assembly as in claim 1, wherein the at least one vacuum source comprises an electric pump in communication with a plurality a separate vacuum ports comprising a first vacuum port in communication with the upper aperture and a second vacuum port in communication with an interior of the container for actively drawing the expressed milk into the container.

13. The breast pump assembly as in claim 3, wherein the at least one vacuum source comprises an electric pump in communication with a plurality of separate vacuum ports comprising a first vacuum port in communication with the upper aperture and a second vacuum port in communication with the opening through the upper portion of the center insert so that each vacuum port comprises a separate vacuum source.

14. A breast pump assembly comprising:
a breast shield having an open proximal end for receiving a breast to form a substantially air-tight seal around the breast and a distal end, the distal end comprising at least one aperture for communicating a suction to an interior of the breast shield;
a conduit extending from the distal end of the breast shield in fluid communication with the breast shield for receiving the expressed milk and directing it to a container;
at least one vacuum source in communication with the aperture for creating a suction within the breast shield; and
a center insert extending outward from the aperture toward the proximal end of the breast shield, the center insert comprising a cylindrical center section with an outer diameter sized to closely fit within the aperture, a flared proximal end having an annular surface for contacting and applying a counterforce against a circular area around a nipple of the breast, and a stepped-down distal section having an outlet end extending into the conduit, the stepped-down distal section having an interior volume much smaller than an interior volume of the cylindrical center portion and a flap valve formed near the outlet to prevent air from being drawn from the container when vacuum is applied while permitting expressed milk to flow into the container, wherein the center section has an opening formed through an upper portion thereof so that the interior volume of the center section is in fluid contact with the at least one vacuum source;
wherein, when a vacuum is applied to the breast shield, the breast and nipple are drawn in toward the distal end of the breast shield while the flared proximal end opposes the inward force to press outward against the breast around the nipple so the milk is expressed into and through the center insert and out into the container.

15. The breast pump assembly as in claim 14, wherein the center insert comprises a silicone gel.

16. The breast pump assembly as in claim 14, wherein the aperture comprises two separate apertures with a partition there between, the two apertures comprising a liquid aperture and a suction aperture disposed above the liquid aperture.

17. The breast pump assembly as in claim 16, wherein the partition between the liquid and suction apertures is arcuate so that the suction aperture partially encircles the liquid aperture.

18. The breast pump assembly as in claim 14, wherein the breast shield has a parabolic shape.

19. The breast pump assembly as in claim 14, wherein the vacuum source is a manual pump comprising a vacuum chamber in fluid communication with the aperture, a flexible diaphragm fitted within and sealing the vacuum chamber, and a handle for moving a center portion of the flexible diaphragm upward and downward relative to the vacuum chamber to generate a vacuum within the vacuum chamber.

20. The breast pump assembly as in claim 19, wherein the handle further comprises a locking mechanism for locking the flexible diaphragm in a raised position.

21. The breast pump assembly as in claim 19, further comprising a pull rod having a lower end attached to the center portion of the flexible diaphragm and an upper end attached to a knob, wherein the handle cooperates with the knob to raise and lower the pull rod.

22. The breast pump assembly as in claim 21, wherein the pull rod has a variable length for adjusting a range of travel of the lower end of the pull rod.

23. The breast pump assembly as in claim 14, wherein the vacuum source is an electric pump.

24. The breast pump assembly as in claim 14, wherein the at least one vacuum source comprises an electric pump in communication with a plurality a separate vacuum ports comprising a first vacuum port in communication with the upper aperture and a second vacuum port in communication with an interior of the container for actively drawing the expressed milk into the container.

25. The breast pump assembly as in claim 14, wherein the at least one vacuum source comprises an electric pump in communication with a plurality of separate vacuum ports comprising a first vacuum port in communication with the upper aperture and a second vacuum port in communication with the opening through the upper portion of the center insert so that each vacuum port comprises a separate vacuum source.

26. A breast pump assembly comprising:
a breast shield having a parabolic shape generally conforming to a shape of a breast with an open proximal end for receiving and forming a substantially air-tight seal around the breast and a distal end for communicating expressed milk to a container, the distal end having at least one aperture;
at least one vacuum source in communication with the at least one aperture for creating a suction within the breast shield; and
a center insert extending outward from the at least one aperture toward the proximal end of the breast shield, the center insert comprising a cylindrical center section with an outer diameter sized to closely fit within an interior of the aperture, a flared proximal end having an annular surface for contacting and applying a counterforce against a circular area around a nipple of the breast, and a stepped-down distal section having an outlet end extending into the conduit, the stepped-down distal section having an interior volume much smaller than an interior volume of the cylindrical center portion and a valve formed therein to prevent air from being drawn from the container while permitting expressed milk to flow into the container, wherein the center section has an opening formed through an upper portion thereof so that the interior volume is in fluid contact with the at least one vacuum source.

27. The breast pump assembly as in claim 26, wherein the center insert comprises a silicone gel.

28. The breast pump assembly as in claim 26, wherein the at least one aperture comprises two separate apertures with a partition therebetween, the two apertures comprising a liquid aperture and a suction aperture disposed above the extraction aperture.

29. The breast pump assembly as in claim 28, wherein the partition between the liquid and vacuum apertures is arcuate so that the vacuum aperture partially encircles the liquid aperture.

30. The breast pump assembly as in claim 26, wherein the at least one vacuum source is a manual pump comprising a vacuum chamber in fluid communication with the aperture, a flexible diaphragm fitted within and sealing the vacuum chamber, and a handle for moving a center portion of the flexible diaphragm into and out of the vacuum chamber to generate a vacuum within the vacuum chamber.

31. The breast pump assembly as in claim 30, wherein the handle further comprises a locking mechanism for locking the flexible diaphragm in a raised position.

32. The breast pump assembly as in claim 30, further comprising a pull rod having a lower end attached to the center portion of the flexible diaphragm and an upper end attached to a knob, wherein the handle cooperates with the knob to raise and lower the pull rod.

33. The breast pump assembly as in claim 32, wherein the pull rod has a variable length for adjusting a range of travel of the lower end of the pull rod.

34. The breast pump assembly as in claim 26, wherein the vacuum source is an electric pump.

35. The breast pump assembly as in claim 26, wherein the at least one vacuum source comprises an electric pump in communication with a plurality a separate vacuum ports comprising a first vacuum port in communication with the upper aperture and a second vacuum port in communication with an interior of the container for actively drawing the expressed milk into the container.

36. The breast pump assembly as in claim 26, wherein the at least one vacuum source comprises an electric pump in communication with a plurality of separate vacuum ports comprising a first vacuum port in communication with the upper aperture and a second vacuum port in communication with the opening through the upper portion of the center insert so that each vacuum port comprises a separate vacuum source.
